(19) United States
(12) Patent Application Publication
(10) Pub. No.: US 2011/0186045 A1
(43) Pub. Date: Aug. 4, 2011

(54) Breathing Apparatus

(76) Inventor: Lenard Erickson, Outlook (CA)

(21) Appl. No.: 13/015,025

(22) Filed: Jan. 27, 2011

Related U.S. Application Data
(60) Provisional application No. 61/299,417, filed on Jan. 29, 2010.

(51) Int. Cl. A61M 16/08

(52) U.S. Cl. 128/201.23; 128/206.28

(57) ABSTRACT

There is a requirement within certain environments to provide an individual with positive pressure air flow or oxygen flow, commonly referred to as Positive Air Pressure (PAP). Such environments including sleeping, racing, and firefighting for example and addressed within the prior art by approaches such as full face masks or inserts into the users nostrils and/or mouth. However, such approaches have limitations in respect of at least one of allowing speech, smell, or motion. According to embodiments of the invention the user is provided with a head covering that provides placement of vents in proximity of the user's nostril and/mouth. The connection from the source of positive pressure, such as pump or pressurized container, includes in some embodiments gimbal elements to provide increased flexibility of user motion.

### FIELD OF THE INVENTION

This invention relates to breathing apparatus and more specifically to providing an improved apparatus for non-invasive provisioning of positive air pressure to a user.

### BACKGROUND OF THE INVENTION

Sleep apnea is a common disorder in which an individual has one or more pauses in breathing or shallow breaths while they sleep; apnea is the combination of the Greek words “a” meaning without and “pnea” meaning air. The resulting loss of air movement during sleep results in a depletion of oxygen and a build up of carbon dioxide in the lungs and blood. Breathing pauses can last from a few seconds to minutes. They often occur 5 to 30 times or more an hour. Typically, normal breathing then starts again, sometimes with a loud snort or choking sound and is accompanied by a movement out of deep sleep and into light sleep when the breathing pauses or becomes shallow resulting in poor sleep quality. Sleep apnea often goes undiagnosed. Doctors usually can't detect the condition during routine office visits. Also, there are no blood tests for the condition. Most people who have sleep apnea don't know they have it because it only occurs during sleep. A family member and/or bed partner may first notice the signs of sleep apnea.

Today in North America it is estimated that between 4%-9% of middle-aged men and between 2%-4% of middle-aged women suffer from sleep apnea. Roughly 30% of the population of the United States are middle-aged, assuming 35-55 as middle aged (US National Census Bureau). As of Dec. 22, 2009 the US population was approximately 308 million people. Hence, between 6.2 million and 13.9 million middle-aged men and between 3.1 million and 6.2 million middle-aged women suffer from sleep apnea in the United States alone. Worldwide these numbers are probably between 3-5 times higher but the true numbers may be significantly higher as between 80%-90% of people in North America with sleep apnea go undiagnosed.

As shown in FIG. 1 with first body section 100 when an individual is awake air flows in and out of their nasal passages and throat. When the individual is asleep, shown in second body section 110, a partial vacuum develops in their throat causing a partial collapse. This vacuum develops when air moves through a restricted space at high speed, resulting in turbulent airflow within the nose and thereby snoring, the most obvious event associated with sleep apnea. The more severe the restriction, the louder the snoring becomes and the more likely apnea is to occur. When the throat collapses like a pinched straw on inspiration, carbon dioxide levels rise and oxygen levels decrease, resulting in arousal as shown in third body section 120. The patient will simply lighten their sleep for a few seconds (arousal), open the airway to breathe and return to sleep, only to resume snoring and repeat the apnea cycle about once per minute. Therefore, in more severe cases, it is possible for a person to experience 500 to 600 episodes of sleep apnea each night. The bed partner becomes aware of this problem because of pauses in air flow that are followed by gasping.

Sleep apnea is a progressive disease, mild snoring converts gradually to sleep apnea as more negative pressures are created because of weight gain, aging, etc. Initially this may be only a few apneas per night but as the number of apneic events increases, both physical and mental symptoms develop. These are usually not noted until there are at least 50 or more events per night. These are several symptoms which indicate the possible presence of apnea including weight gain, fitful sleep, tiredness, loud snoring, mumbling in sleep, and drooling. Cognitive dysfunction may also be present including poor concentration, poor memory, irritability, chronic fatigue, decrease libido, depression, and claustrophobia. Long-term effects of sleep apnea include increased risk of cardiovascular disease, heart attack, stroke, angina pectoris (chest pains) and hypertension. Whilst symptoms may vary amongst sufferers it is clear that these are increasingly severe as the apnea continues.

Typically those with sleep apnea are encouraged to address the issue through lifestyle changes to address issues such as weight and smoking. However, three of the major risk factors for sleep apnea are largely determined at birth, being age as prevalence and severity of symptoms increase with age, gender, as men are about twice as likely to develop sleep apnea until women reach menopause, and airway size and shape where factors including cranial structure, small jaw, large tongue, large tonsils, and narrow airway. Other factors include diseases such as emphysema, asthma, neuromuscular disease, nasal obstruction and hypothyroid. As a result lifestyle changes generally do not address the problem resulting in the need for prosthetic devices or ultimately surgical interventions. Such surgical interventions include nasal surgery, plastic surgery of palate, uvula and pharynx, jaw relocation, facial surgery and tracheotomy.

As a result intervention in sleep apnea is dominated by prosthetic devices as sufferers do not wish to result in the more severe and drastic surgical interventions. The most frequently used treatment for sleep apnea is positive airway pressure (PAP), primarily continuous PAP (CPAP). In this therapy, a prosthetic device consisting of an airflow generator, a flexible hose and a mask is attached to the sufferer whilst they sleep, such as shown in FIG. 2 wherein air is provided to the mask 240 through air pipe 220. The mask 240 being held in place on the sufferer's head 210 through straps 230. CPAP uses air under pressure to splint the sufferers airway passage open and prevents both snoring and obstruction of airflow in and out of the lungs. CPAP has several advantages including eliminating apnea, being effective on almost all patients, quick, relatively cheap, and non-invasive.

However, most masks for CPAP are based primarily upon surgical devices and are linked to the airflow generator with large pipes that come directly to the mask. For most suffers they are initially reluctant to use the therapy, as the nose mask and hose to the airflow generator look uncomfortable and clumsy, and indeed they are. In fact a significant number of sufferers will discontinue using them due to the discomfort, restricted sleeping position, etc that the masks of the prior art require. Further other sufferers take a long period of time to adjust to the treatment. Coupled with this there are a significant number of PAP manufacturers who offer different models at different price ranges, and PAP masks have many different sizes and shapes, so that users may need to try several masks before finding a good fit. These different machines may not be comfortable for all users and the purchase, evaluation of these is clearly expensive and frustrating to the sufferer irrespective of the benefit they derive and selection of PAP models may be very important in furthering adherence to therapy. Beards, mustaches or facial irregularities may interfere also with the mask, and where the mask contacts the skin it must be free from dirt and excess chemicals (such as skin oils). For me shaving before mask-fitting may be necessary. Further PAP masks and restraints according to the prior art are of limited designs and essentially utilitarian with no ability for the sufferer to personalize, enhance, or disguise the mask.

It would, therefore, desirable to provide users with a PAP facial device that has reduced issues for the user in terms of being compatible with facial irregularities, beards, mustaches, chemicals as most female users will use face creams, cleaners, night masks etc. It would be further beneficial if the PAP facial device was less cumbersome, less restrictive on sleeping position and allows the user the opportunity to personalize or disguise the mask. Further it would be beneficial for the device not to have to insert tubes into their nostrils, insert mouthpieces or combinations thereof. Accordingly it is an aspect of the invention to provide a PAP with said benefits.

### SUMMARY OF THE INVENTION

It is an object of the present invention to obviate or mitigate at least one disadvantage of the prior art.

In accordance with an embodiment of the invention there is provided a device comprising a shell comprising at least an outer layer covering a predetermined portion of a users head, a first pipe, a connector attached to a predetermined location of the shell and for coupling a first end of the first pipe to a second pipe, and a first vent attached to a predetermined location of the shell and comprising a first inlet and a first outlet, the first outlet disposed in a predetermined location relative to at least one of the users mouth and the users nostril and the first inlet coupled to second end of the first pipe.

In accordance with another embodiment of the invention there is provided a device a vent piece for demountably attaching to a vent, the vent piece comprising at least one opening of a plurality of openings to couple a gas flow within the vent piece to outside the vent piece, wherein the vent forms part of a shell comprising at least an outer layer covering a predetermined portion of a users head, a first pipe, a connector attached to a predetermined location of the shell and for coupling a first end of the first pipe to a second pipe, and the vent which is attached to a predetermined location of the shell and comprising a first inlet and a first outlet, the first inlet coupled to second end of the first pipe and the first outlet disposed in a predetermined location relative to at least one of a users mouth and a users nostril when the user wears the shell and none of the vent piece, the plurality of openings, and shell enclose the at least one of the users mouth and the users nostril.

In accordance with another embodiment of the invention there is provided a head gear comprising covering a predetermined portion of a users head and comprising at least an outer layer, an inlet port for accepting gas and at least an outlet port of a plurality of outlet ports for venting the gas and a a connector attached to the inlet port for coupling the device to the source of gas; wherein attaching at least one vent of a plurality of vents to the at least an outlet port provides a venting of the gas under pressure at a predetermined location relative to at least one of the users mouth and the users nostril and where none of the head gear and vent enclose the at least one of the users mouth and the users nostril.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

Embodiments of the present invention will now be described, by way of example only, with reference to the attached Figures, wherein:

FIG. 1 depicts the stages of sleep apnea;

FIG. 2 depicts a schematic of a face mask according to the prior art;

FIG. 3 depicts a Hans Rudolph 7600 Series V2 CPAP mask according to the prior art;

FIG. 4 depicts a series of PAP and CPAP masks according to the prior art;

FIG. 5 depicts a PAP head gear according to an embodiment of the invention;

FIG. 6 depicts a PAP head gear according to an embodiment of the invention with increased air circulation to the head;

FIG. 7 depicts a PAP head gear according to an embodiment of the invention with air vents disposed relative to nose and mouth of the sufferer;

FIG. 8 depicts a PAP head gear according to an embodiment of the invention with dual air vents and personalized to the sufferer;

FIG. 9 depicts a PAP head gear according to an embodiment of the invention with personalized masks and a nasal bridge in front of the sufferer's nose;

FIG. 10 depicts a PAP head gear according to an embodiment of the invention wherein nasal and mouth bridges in front of the sufferer's face;

FIG. 11 depicts PAP systems according to embodiments of the invention introduced to safety helmets;

FIG. 12A depicts a sectional view of PAP head gear according to an embodiment of the invention;

FIG. 12B depicts a PAP head gear according to an embodiment of the invention wherein the head gear forms the air transport without requiring specific tubing within the head gear; and

FIG. 13 depicts PAP head gear according to an embodiment of the invention wherein the head gear is designed to resemble another form of head gear.

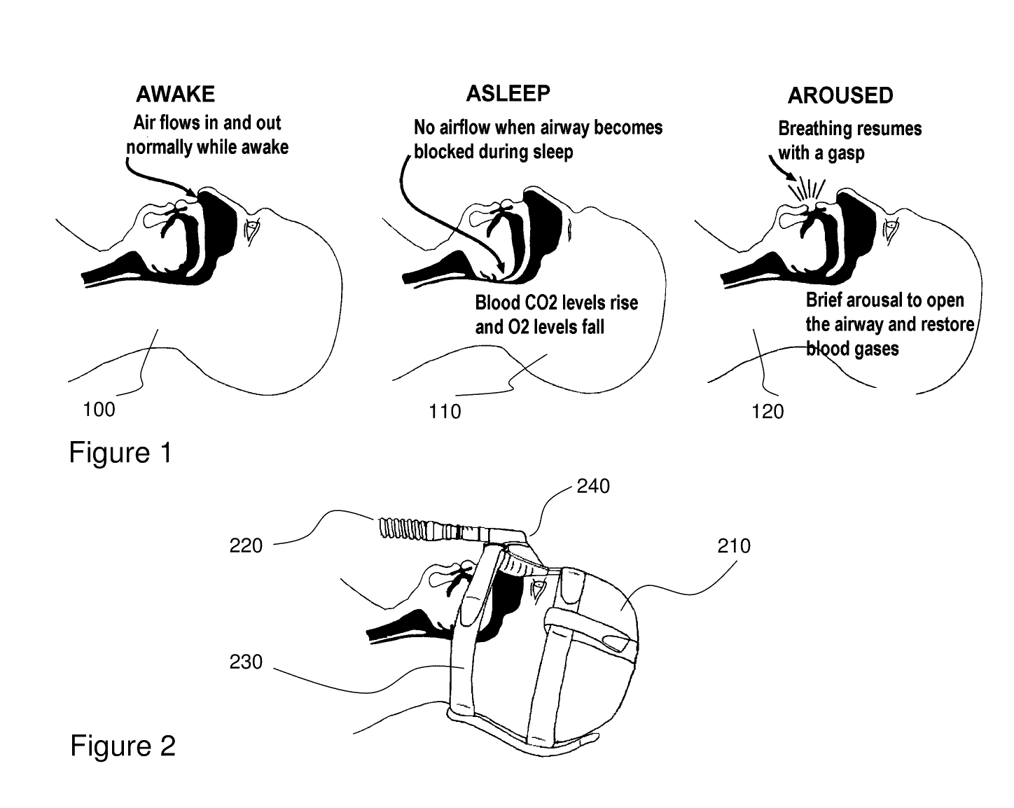

### DETAILED DESCRIPTION

The present invention is directed to providing breathing apparatus for individuals with increased flexibility and maneuverability whilst providing positive air pressure for medical applications such as the treatment of sleep apnea and emergency applications such as firefighters.

Reference may be made below to specific elements, numbered in accordance with the attached figures. The discussion below should be taken to be exemplary in nature, and not as limiting of the scope of the present invention. The scope of the present invention is defined in the claims, and should not be considered as limited by the implementation details described below, which as one skilled in the art will appreciate, can be modified by replacing elements with equivalent functional elements.

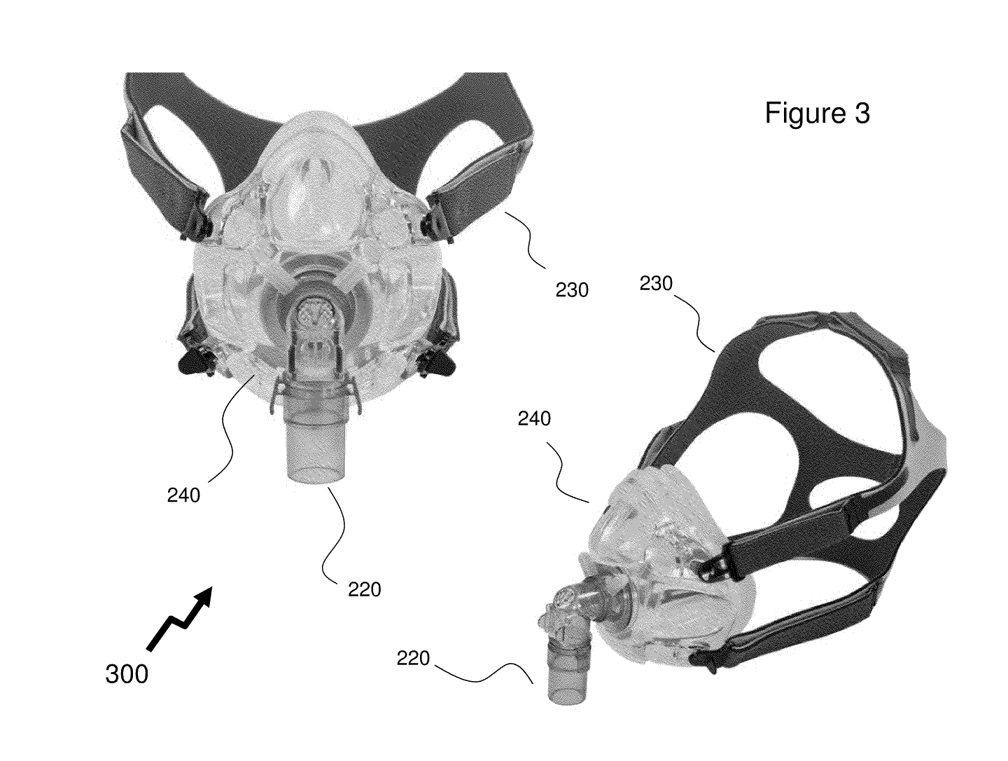

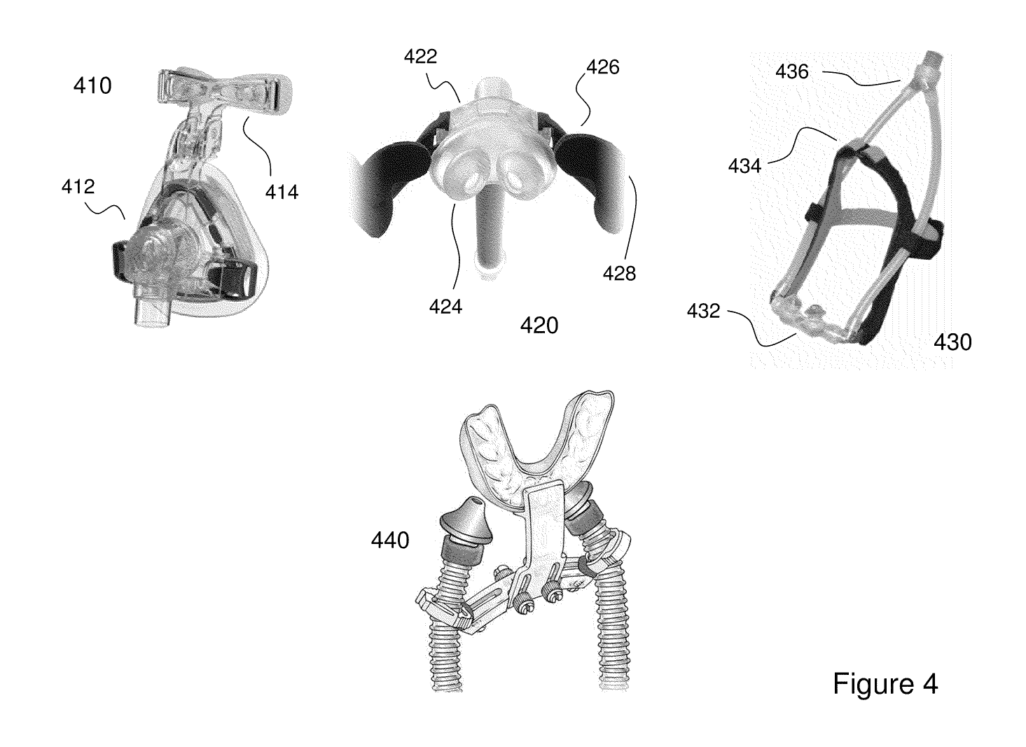

Referring to FIG. 3 there is shown a CPAP mask 300 according to the prior art such as described supra in respect of FIG. 2 with mask 240, straps 230 and air pipe 220. Mask 240 covers the sufferer's nose and is retained in position with respect to their face by straps 230. Air under positive pressure at the prescribed airflow for the sufferer is coupled from a pump (not shown for clarity) to the mask 240 via an air hose (not shown for clarity) which connects to the air pipe 220. Within FIG. 3 CPAP mask 300 is a Hans Rudolph 7600 Series V2 Full Face CPAP Mask and comprises silicone rubber for the mask 240 and polycarbonate for the air pipe 220 and the swivel port connection between the air pipe 220 and mask 240. The air pipe 220 and mask 240 are designed to be cold chemical, steam autoclave, pasteurization, and dishwasher safe. Currently the Hans Rudolph 7600 retails for approximately US$170 to the sufferer without air hose, pump etc.

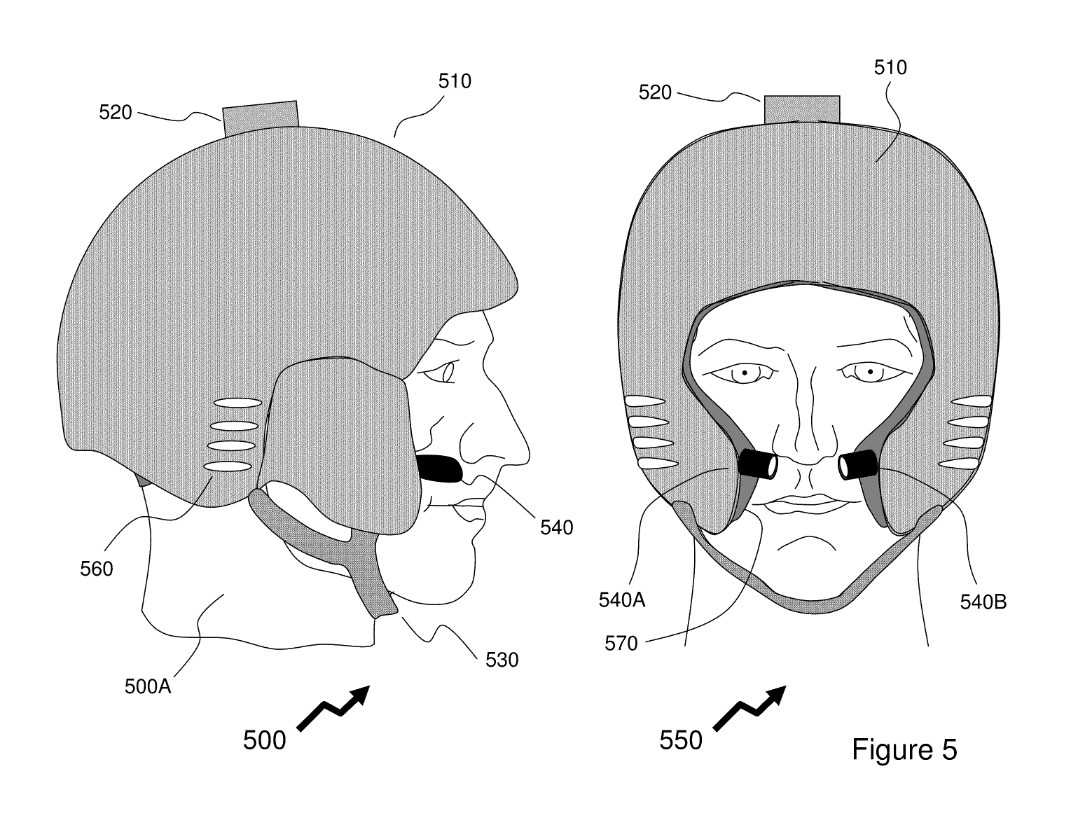

Referring to FIG. 4 other CPAP/PAP masks according to the prior art are shown including ResMed Activa 410, ResMed Swift 420, Bravo 430 and OA/CPAP 440. ResMed Activa 410 comprises a mask 412 to provide air flow to the nose and mouth and forehead stabilization 414 which is intended to maintain placement of the mask 412 when ResMed Activa 410 is placed over the sufferer's face and retained with straps, not shown for clarity. The ResMed Swift 420 comprising body 422 which accepts the incoming air and directs this to the two nasal inserts 424 which are inserted into the sufferer's nostrils. The ResMed Swift 420 being held in place on the sufferer's head by strap 428 and positioned relative to their nostrils by cheek mounts 426. Bravo 430 comprises body 432 with nasal inserts and is retained in position by head-strap 434 differs from ResMed Mirage 410 and ResMed Mirage 420 in that the position of the air pipe 436 is connected to the air generator above the head of the sufferer rather than at the front of their face. In some instances the sufferer may wish to avoid the straps and restraints employed in the prior CPAP and PAP masks.

Accordingly in some instances such as shown by PAP mask 440 there is a mouthpiece providing jaw stabilization of the nasal inserts for the sufferer, the mouthpiece in some sufferers improving alignment of their upper and lower jaws and reducing other aspects of sleep apnea such as snoring. Bravo 430 ResMed Mirage 410 is a ResMed Mirage Activa CPAP Mask currently retailing for approximately US$200, ResMed Swift 420 is a ResMed Mirage Swift LT for Her CPAP mask currently retailing for approximately US$175, and the Bravo 430 is a Bravo CPAP Mask Pillow System retailing for approximately US$100.

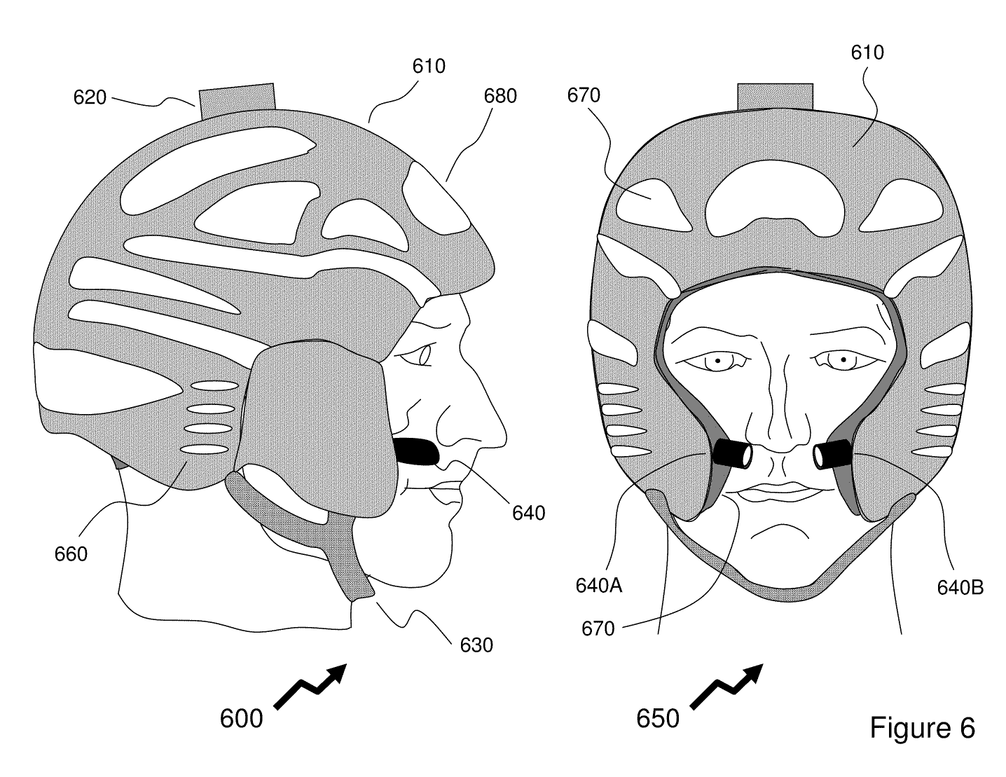

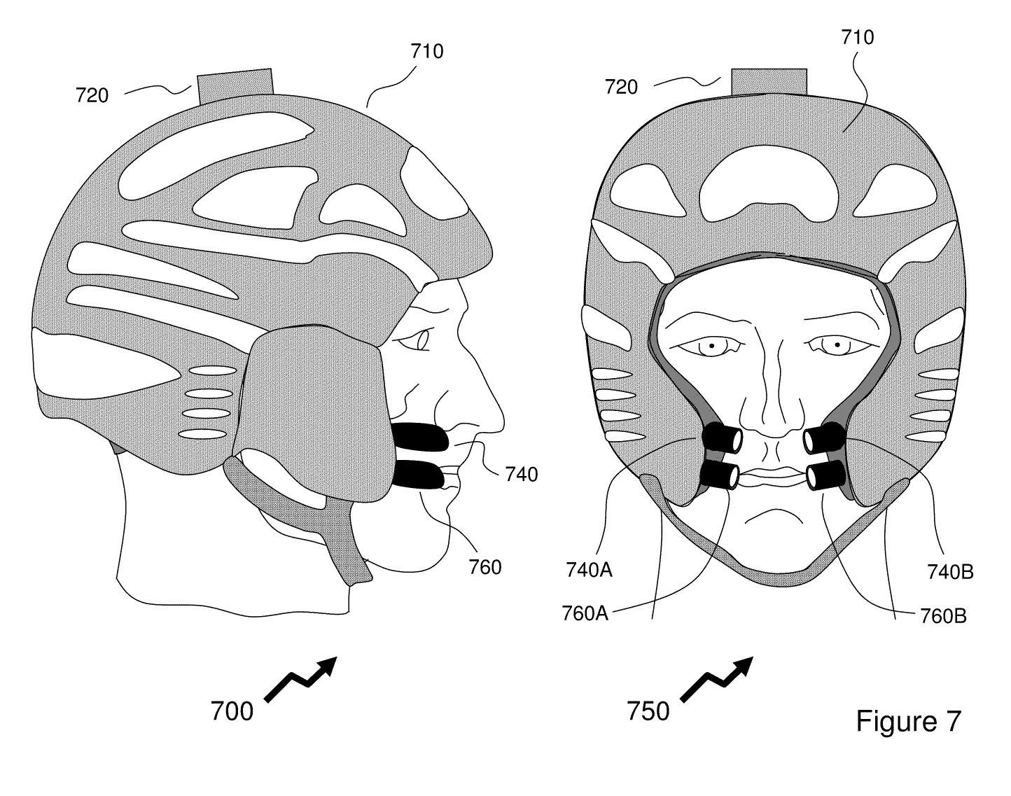

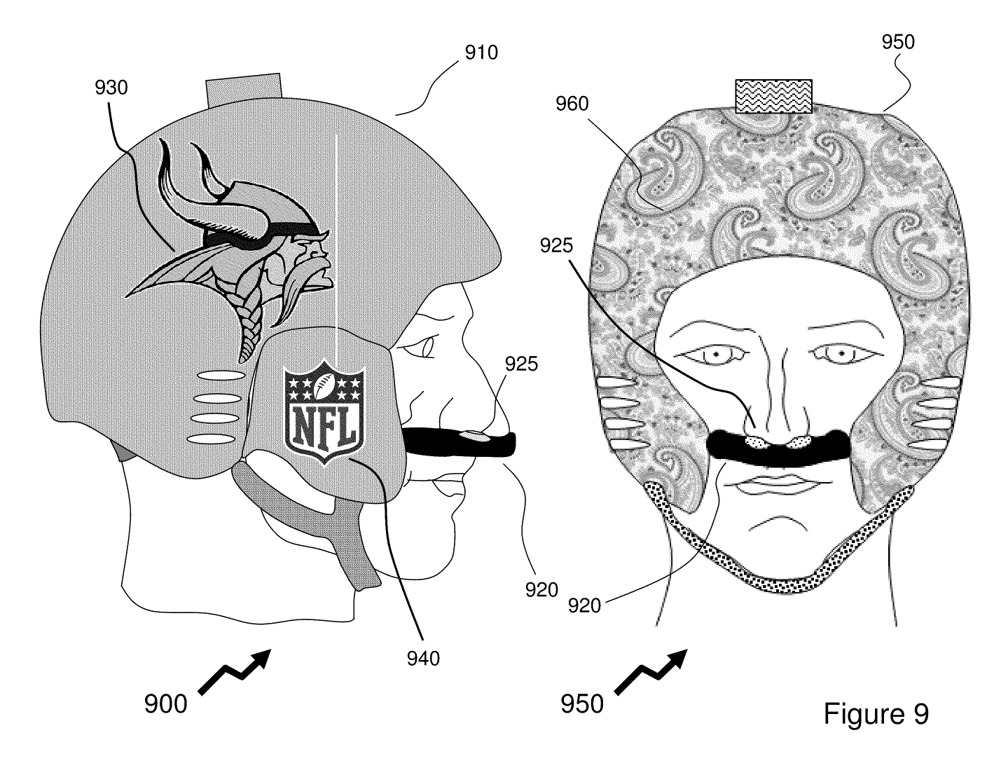

Referring to FIG. 5 there is shown PAP headgear according to an embodiment of the invention comprising side view 500 and front view 550. The user is shown wearing head covering 510 which surrounds a predetermined portion of the user's head 500A and is retained in position by chin strap 530. As shown in side view 500 the head covering 510 has a series of openings 560 which are disposed proximate to the user's ears allowing them clear hearing whilst wearing the head covering 510. Shown at the top of the head covering 510 is connection 520 which provides connection to the remote air pump, not shown for clarity. Also shown disposed proximate the user's nose is outlet 540 which is connected to the connection 520 via tubing which is disposed within the head covering 510 and is not shown explicitly within the embodiments presented here in respect of FIG. 5 and subsequent FIGS. 6 through 11. Referring to front view 550 it is shown that outlet 540 in side view 500 is actually a pair of vents 540A and 540B which are disposed to either side of the user's nose in proximity to their nostrils. The vents 540A and 540B are embedded within the lining 570 of the head covering 510. As would be evident to one skilled in the art head covering 510 with vents 540A and 540B provides a more comfortable PAP mask for the sufferer as the air pumped from the remote pump is dispensed proximate their nose rather than through fittings inserted into their nostrils or via a mask body that covers their noses and mouth. As such the sufferer is able to communicate normally whilst falling asleep/waking etc but deriving benefit from the PAP mask. It would be evident to one skilled in the art that the vents 540A and 540B cause pressurized air to be directed into the user's nostril. As such it would be beneficial to provide a seal between vents 540A and 540B and the head covering 510. Optionally the vents 540A and 540B may be shaped to direct the airflow to the sufferers nostrils.

Now referring to FIG. 6 there is shown PAP head gear according to an embodiment of the invention as shown in side view 600 and front view 650. As shown in side view 600 the PAP mask is shown as a head covering 610 that surrounds a predetermined portion of the user's head and is retained via chin strap 630. Connection to the remote pump providing the positive air pressure is via connection 620 at the top of the head covering 610. As the connection 620 is disposed to the top of the user's head then when they are laying down in bed as they turn in their sleep or move the air hose connecting the PAP head gear does not interfere or restrict their motion. It also does not come between the user and a partner in bed or cause issues associated therefrom. As with PAP head gear supra in respect of FIG. 5 the PAP head gear is shown as providing vent 540 in proximity to the user's nose in side view 600 which is actually two airways 640A and 640B embedded within the lining 670 of the head covering 610. As before the head covering 610 has first openings 660 disposed proximate to the user's ears but now the head covering 610 comprises a plurality of second openings 680 which allow for example the weight of the head covering 610 to be reduced or reduce sweating.

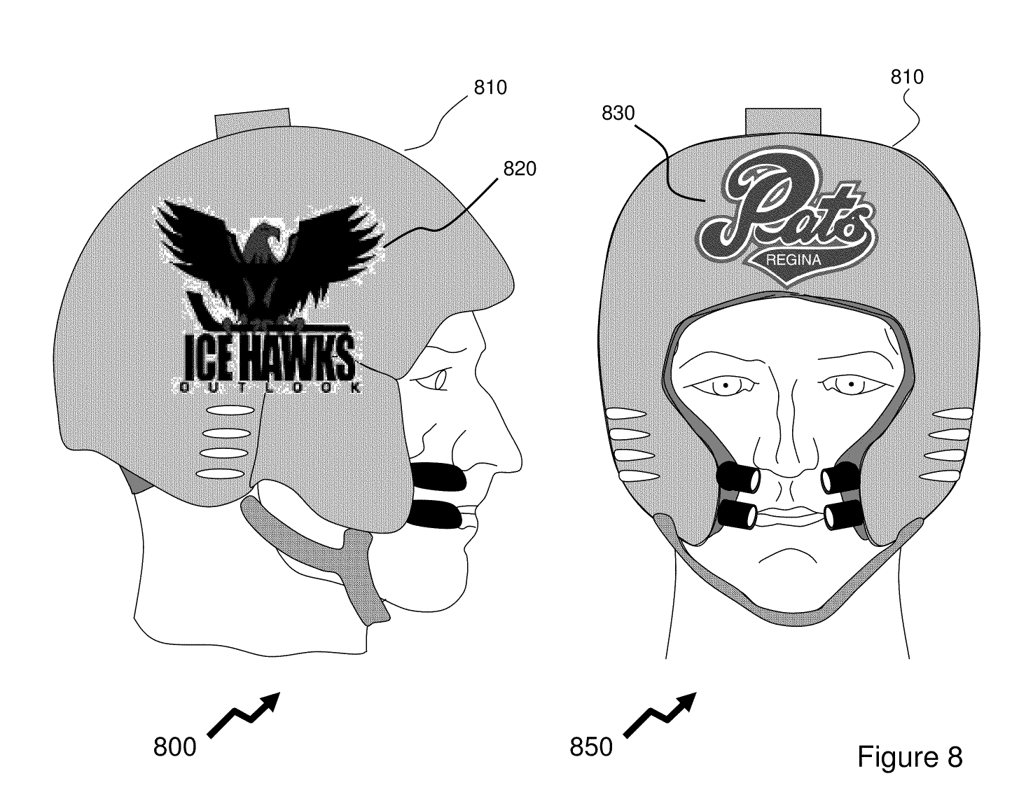

Now referring to FIG. 7 there is PAP head gear according to an embodiment of the invention wherein again as shown in side view 700 the positive air pressure to the sufferer is provided by first vent 740 disposed proximate the sufferer's nose and second vent 760 disposed proximate their mouth. As is evident from front view 750 the first vent 740 of side view 700 is actually two nose vents 740A and 740B within the head covering 710. Similarly the second vent 760 of the side view 700 is actually two mouth vents 760A and 760B within the head covering 710. The two nose vents 740A and 740B as well as the two mouth vents 760A and 760B are connected to the connection 720 disposed to the top of the head covering 710.

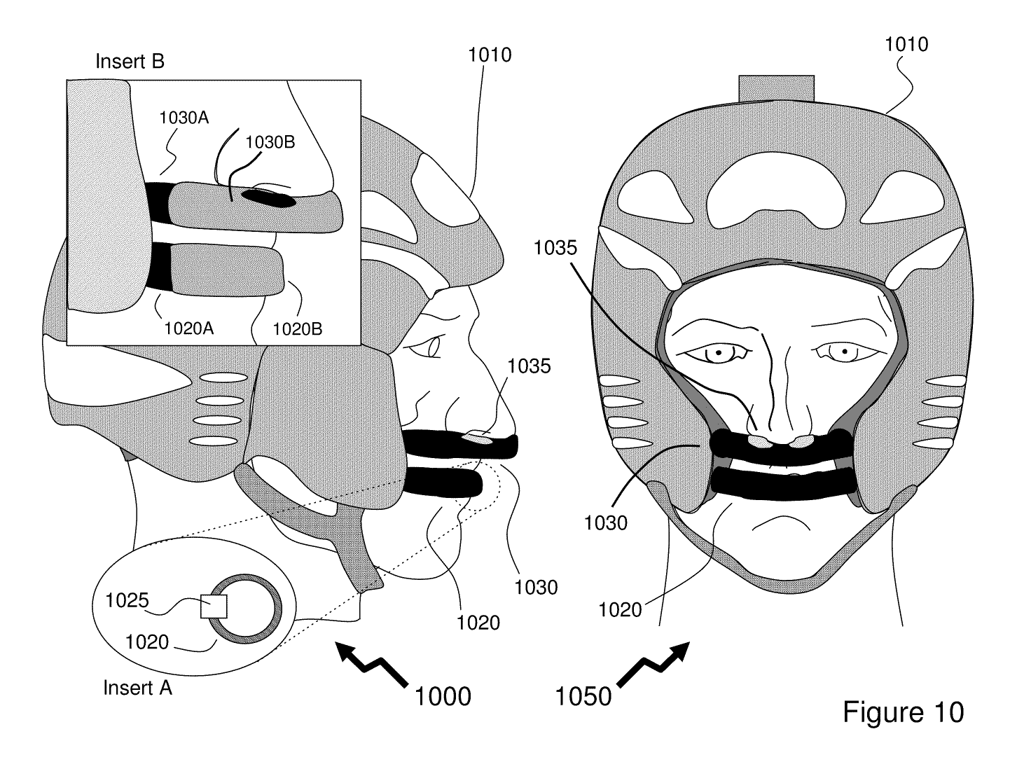

It would be apparent that the head coverings 510, 610 and 710 described supra in respect of FIGS. 5, 6 and 7 respectively that the outer covering of the head coverings 510, 610 and 710 would be predetermined by the supplier of the PAP head gear. As such the selections may be limited or may be lacking personality. As shown in side view 800 the head covering 810 has a logo 820 disposed upon it, in this example representing the Outlook Ice Hawks, a senior league hockey team being part of the Saskatchewan River Valley Hockey League. In front view 850 the head covering 810 having a logo 830 disposed upon it, in this example representing the Regina Pats, a junior hockey league team being part of the Western Hockey League in Canada. The material of the head covering may be patterned prior to delivery to the sufferer or may be selected from a material that allows a user to choose and either print themselves or have printed a pattern that can be ironed or affixed to the outer surface of the head covering. Additional examples of head coverings of the PAP head gear are shown in FIG. 9 with first head gear 910 in side view 900 and second head gear 950 in front view 950. As shown in first head gear 910 the sufferer is a fan of the National Football League (NFL) and has the logo 940 of the NFL on the side of their PAP head gear as well as the team logo 930, in this case of the Minnesota Vikings. As shown in second head gear 950 the sufferer, in this case most likely a woman, has chosen a paisley pattern 960 for the covering. Unlike the head coverings 510, 610, 710 and 810 presented supra head gear 910 and 950 provide a nasal air vent 920 to the user which runs under the nose of the sufferer. In the upper surface of the nasal air vent 920 are openings 925 that release under the nostrils of the sufferer.

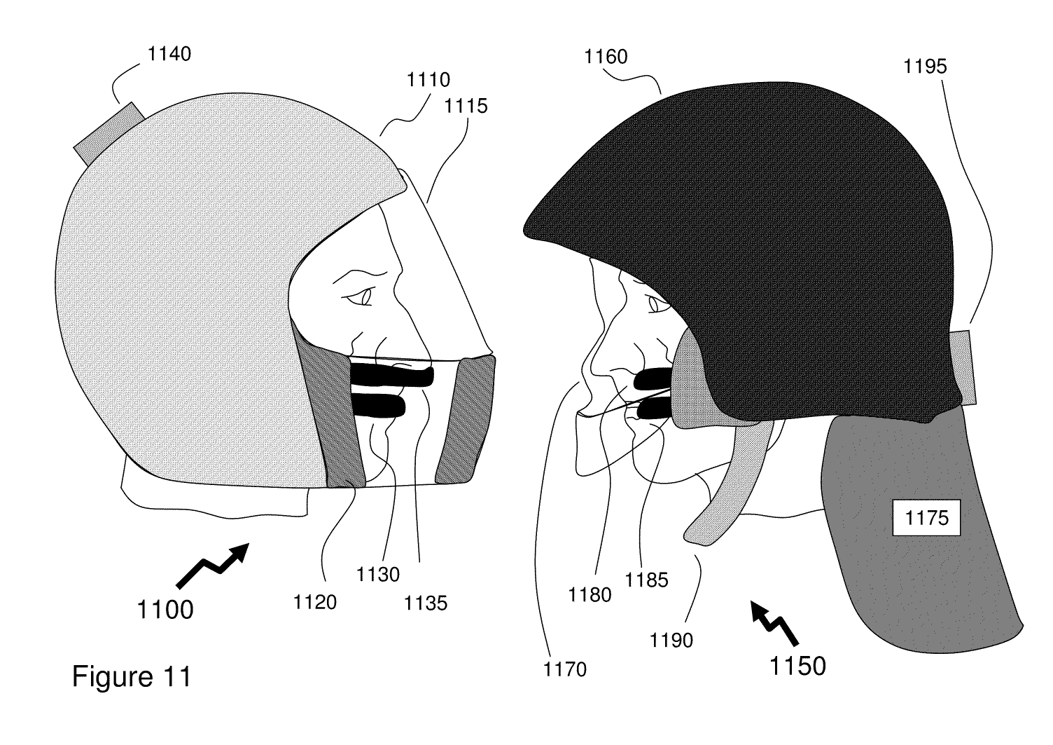

Now referring to FIG. 10 there is shown another embodiment of the invention showing PAP head gear 1010 which has the continuous nasal air vent 1030 across the face of the sufferer with nasal openings 1035. Also shown is continuous mouth air vent 1040 which runs across the face of the user in front of their mouth. A cross-section of mouth air vent 1020 in front of the sufferer's mouth is shown in Insert A showing a mouth opening 1025 within the tubing forming mouth air vent 1020. It would be evident to one skilled in the art that the nasal air openings 1035 and mouth air openings 1035 may be a plurality of small holes rather than the single large openings depicted or implied within the nasal air vent 1030 and mouth air vent 1020. Optionally these may be disposable or replaceable tubing which mounts to fittings within the PAP head gear 1010 at the sides near the sufferer's nose and mouth such as shown in Insert B of FIG. 10. As shown in Insert B nasal air vent 1030 comprises a nasal mounting 1030A to which nasal tubing 1030B is attached. Similarly mouth air vent 1020 comprises mouth mounting 1020A and mouth tubing 1020B.

It would be evident to one skilled in the art that other designs of nasal vents are possible to those described above in respect of FIGS. 5 through 10. Whilst the embodiments discussed supra have been those not invasive to the user's nostrils or mouth other may be. Optionally such nasal or mouth insertion elements may be demountably attached to the embodiments presented supra such that they may be removed and cleaned, changed etc.

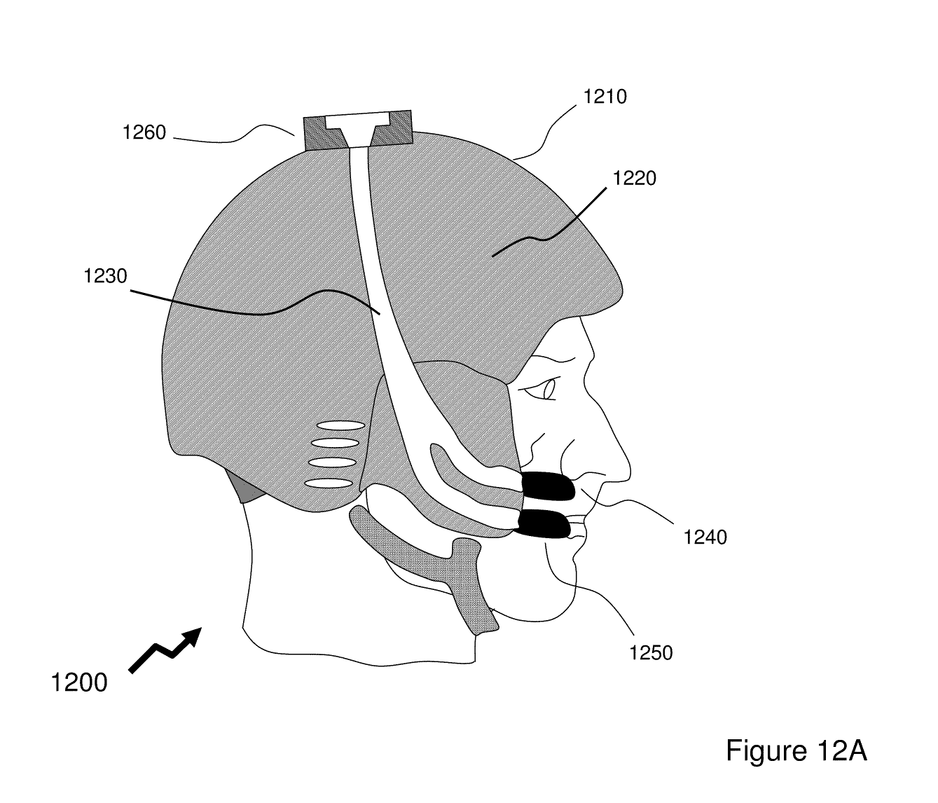

The provisioning of air or oxygen to a user under positive pressure relative to their surroundings is not unique to sufferers of sleep apnea. Referring to FIG. 11 there is shown first helmet 1100 and second helmet 1150. First helmet 1100 for example may be employed by a driver such as within National Association for Stock Car Auto Racing (NASCAR), Formula One (F1), Touring car and rallying for example and comprises body 1110, air pump connection 1140, and visor 1110. Shown in part section view X-X in the region of first helmet 1100 are shown lining 1120, mouth vent tube 1135, and nasal vent tube 1130. Mouth vent tube 1135 and nasal vent tube 1130 for example being similar to those shown supra in respect of FIG. 10. Second helmet 1150 for example being a firefighter safety helmet comprising air pump connector 1195, body 1160, visor 1170, chin strap 1190, and neck guard 1175. Positive air or oxygen pressure to the firefighter is provided by nasal vent tube 1180 and mouth vent tube 1185, which are for example similar to those presented supra in respect of FIG. 10.

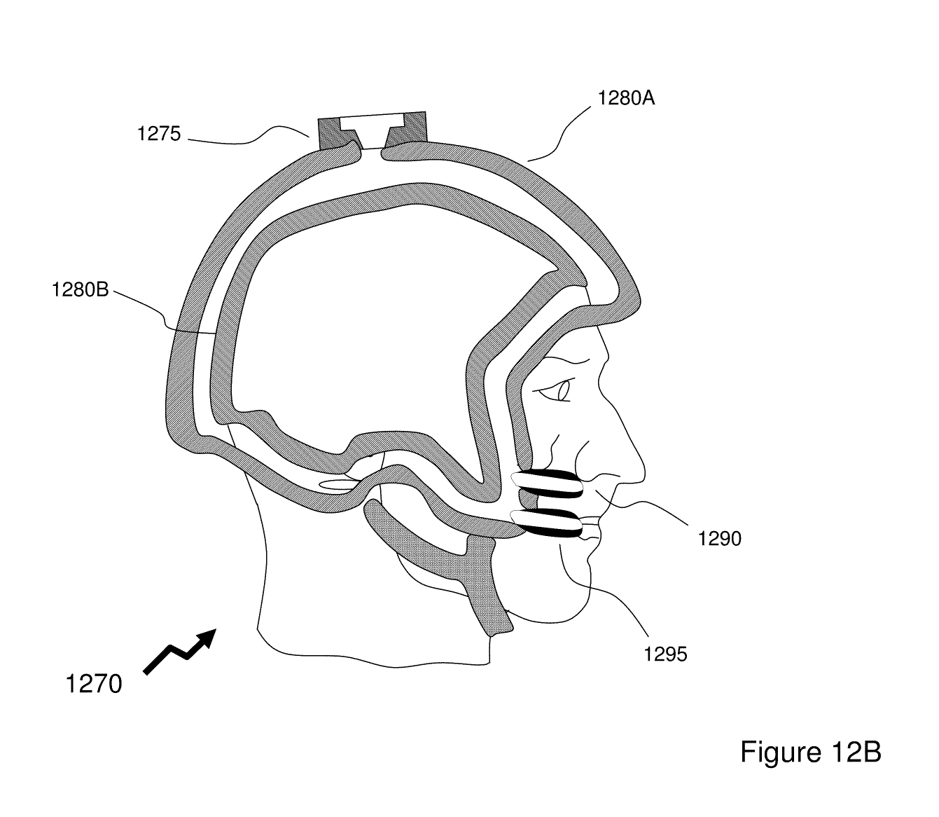

Referring to FIG. 12A there is depicted a cross-section of a PAP head gear 1200 according to an embodiment of the invention wherein the user has mouth vent 1250 and nasal vent 1240. These are connected to airway tubing 1230 that links back to an airway connector 1260, which would be linked to an air pump, not shown for clarity, to provide the positive air flow to the sufferer. Airway tubing 1230 is embedded within filling 1220 which lines the body 1210 of the PAP head gear 1200.

Within the embodiments presented supra in respect of PAP head gear in FIGS. 5 through 11 the interconnection between the particular vents or bridges providing positive air pressure at predetermined locations relative to the user's face and the air inlet were not described with any particular detail. This was because several interconnection formats could be employed. As noted in FIG. 12A supra on such interconnection is tubing positioned within the head gear. Alternatively the tubing could be formed by leaving voids within a filling forming part of the inner of the head gear such as a lightweight injection mouldable foam or plastic for example. Alternatively the interconnection may be via the multiple holes and pathways within a lightweight injection mouldable porous foam or plastic.

Referring to FIG. 12B there is depicted a PAP head gear 1270 according to an embodiment of the invention wherein air under pressure is presented at inlet 1275 which is connected to a remote source of air under pressure. From inlet 1275 the air flows into a bladder assembly comprising outer wall 1280A and inner wall 1280B. The only other openings in the bladder being located at the positions where nasal vent 1290 and mouth vent 1250 are positioned. As shown the mouth vent 1295 and nasal vent 1290 are inserted into the openings within the bladder. It would evident to one skilled in the art that the openings may be formed with mechanical fittings, such as threaded portions for example allowing the mounting of the nasal vent 1290 and mouth vent 1295 mechanically by threaded connection. Such a demountable connection allowing replacement of the nasal vent 1290 and mouth vent 1295 as appropriate by the user for cleaning, replacement, etc. As such PAP head gear 1270 provides a cushioned air pillow around the user's head as well as providing the air flow mechanism from the inlet 1275 to the nasal and mouth vents 1290 and 1295 respectively.

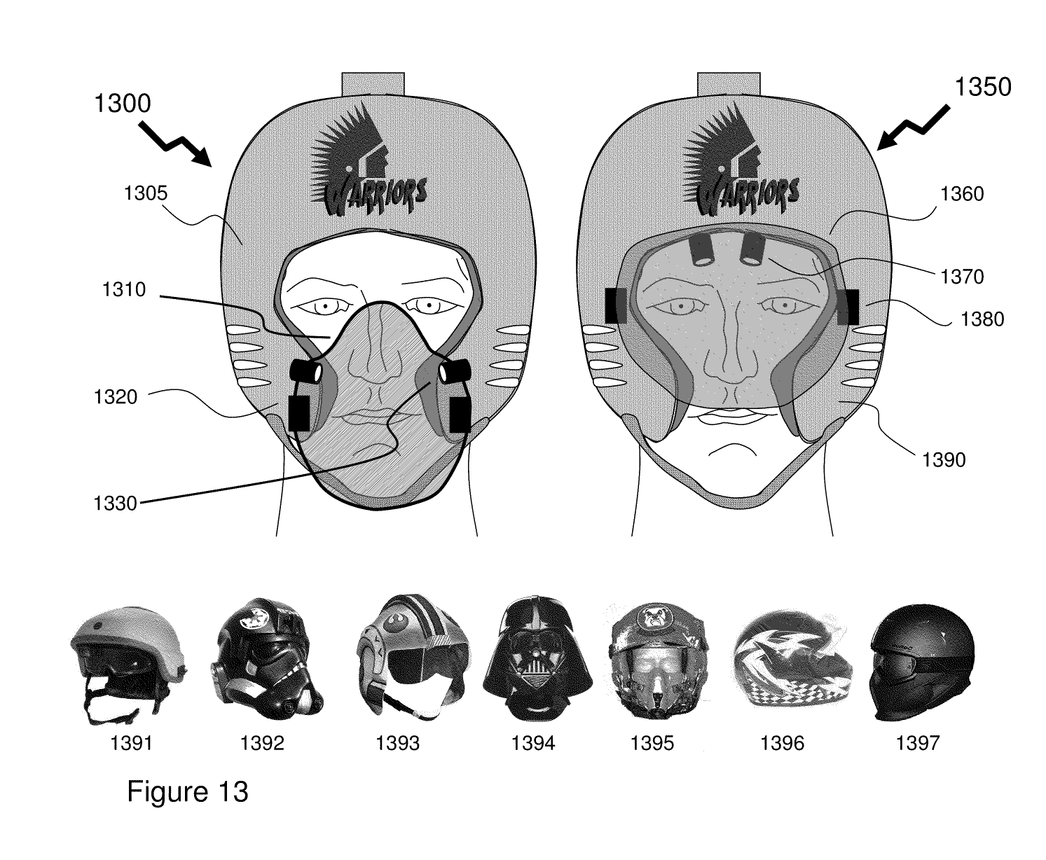

Referring to FIG. 13 there are shown first and second PAP head gear 1300 and 1350 respectively. In first head gear 1300 a mask 1310 is shown covering the lower portion of the user's face and being attached to the head covering 1305 by attachment means 1320, for example including but not limited to a VelcroTM mounting, a snap fitting, and a strap and clip. Also shown are vents 1330 which then provide air within the mask 1330 attached to the head covering 1305. Mask 1310 thereby directs the air flow to around the user's nose and mouth. In second head gear 1350 an alternate mask 1360 is shown covering the user's upper portion of their face and again being attached by fittings 1380 to head gear covering 1390. However in this embodiment the vents 1370 are disposed at the forehead of the user and air flow is directed by the alternate mask 1360 to be around the user's nose. It would be evident to one skilled in the art that the head coverings 1305 and 1390 in conjunction with particular designs of mask 1330 and alternate mask 1360 would allow the user to have a PAP mask that mimicked another mask or helmet design such as for example army helmet 1391, Star Wars™ Imperial fighter pilot 1392, Star WarsTM Rebel fighter pilot 1393, Star Wars™ Darth Vader™ helmet 1394, jet fighter pilot 1395, NASCARTM helmet 1396 and ski racing helmet 1397. Such designs for example making PAP head gear fun for many to use, especially children.

Within the embodiments presented supra in respect of PAP hear gear in FIGS. 5 through 13 the air tubing between the connector and the vents proximate the user's nose or mouth have been described as being embedded within the lining of the head gear. It would be apparent to one skilled in the art that rather than simple tubing the PAP head gear may include additional elements such as air flow regulators, control valves etc. Whilst the sufferer or user may control the air flow through the settings of the air pump to which their PAP head gear is connected it may be beneficial for the PAP head gear to include controls allowing the user to adjust or start and stop the air flow. This may be achieved directly by the user with air flow regulators; control valves etc or alternatively the PAP head gear may contain electrical controls that control the air flow within the PAP head gear or at the air pump. Electrical interconnection from the PAP head gear to the air pump being for example via electrical cabling forming part of the air way connection between the PAP head gear and air pump.

Within CPAP/PAP masks according to the prior art the air pipe connection to the mask is a rigid joint. However, it would be beneficial for this joint, such as airway connection 1260 and connections 520, 620 and 720 in FIGS. 10, 5, 6 and 7 respectively, was a flexible gimbal joint allowing the user's head to move freely as they sleep, drive, etc. Such a flexible gimbal joint may be formed for example in the tubing joint, see for example K. B. Bredtschneider in U.S. Pat. No. 2,862,729 entitled “Flexible Bellows Seal for Flanged Pipe Joint” and M. Uegane et al in U.S. Pat. No. 6,419,280 entitled “Exhaust Pipe Joint Assembly”.

It would be apparent to one skilled in the art that whilst embodiments presented supra in respect of FIGS. 5 through 13 employ chin straps for example that the design of the PAP head gear in encompassing a portion of the user's head may on it's own be sufficient in many instances to maintain the PAP head gear in the correct position as the user moves.

It would be apparent to one skilled in the art that the PAP head gear described supra in respect of FIGS. 5 through 13 does not comprise elements which are inserted into or block either the mouth or the nose of the sufferer or user. Accordingly, the sufferer or user is able to freely talk whilst the PAP head gear is in place, for sufferers this allows them to communicate to a partner for example until they are asleep or in the event of safety helmets communicate freely to others directly around them or via a microphone for example. Similarly in designs where the vents are not blocking their nostrils sufferers may be able to smell, for example, smoke allowing them potentially to react to dangerous circumstances.

It would be evident to one skilled in the art that the external source of positive gas pressure may be other than a pump, such external sources may include compressors and pressured gas containers such as air bottles or oxygen bottles for example. Further whilst the shape of the PAP head gear has within the embodiments for sleep apnea sufferers etc in respect of FIGS. 5 through 10 been considered as having a form that generally conforms to the users head it would be apparent from the safety helmets 1100 and 1150 in FIG. 11 that other shapes may be provided for or by the user. Accordingly the user may wish to employ PAP head gear shaped with respect to a favorite hobby for example and to have it in the shape of a racing driver's helmet, football, or hockey puck. Other options would be evident to one of skill in the art including providing additional elements including but not limited to microphones, headphones, an audiovisual media player, eye shades, eye coverings, face masks, and sun visors. Further whilst the descriptions have been presented in respect of air under pressure it would be evident that this may alternatively be oxygen, premixed oxygen and nitrogen, air or oxygen with airborne medication etc. Further the source of the positive pressure air flow may be an air pump as described but may also include compressed gas cylinders or canisters such that the entire assembly is not only wearable but also portable allowing use in emergency, rescue and other environments.

The above-described embodiments of the present invention are intended to be examples only. Alterations, modifications and variations may be effected to the particular embodiments by those of skill in the art without departing from the scope of the invention, which is defined solely by the claims appended hereto.

What is claimed is:

1. A device comprising:
a shell comprising at least an outer layer covering a predetermined portion of a users head;
a first pipe;
a connector attached to a predetermined location of the shell and for coupling a first end of the first pipe to a second pipe; and
a first vent attached to a predetermined location of the shell and comprising a first inlet and a first outlet, the first outlet disposed in a predetermined location relative to at least one of the users mouth and the users nostril and the first inlet coupled to second end of the first pipe.

2. A device according to claim 1 wherein,
none of the shell, first vent and first outlet enclose at least one of the users mouth and the users nostril.

3. A device according to claim 1 further comprising;
a second vent attached to a predetermined location of the shell and comprising a second inlet and a second outlet, the second outlet disposed in a predetermined location relative to the other of the at least one of the users mouth and the users nostril and the second inlet coupled to at least one of a third end of the first pipe and a third pipe.

4. A device according to claim 1 further comprising;
a control attached to a predetermined location of the shell and for providing the user with at least one of a regulating and a switching function in respect of a gas flow to the first vent.

5. A device according to claim 4 wherein,
the control provides the at least one of function to at least one of the first vent, the first pipe, the second pipe, the connector, and a remote pump.

6. A device according to claim 1 further comprising;
a vent piece demountably attached to at least the first vent and comprising at least one opening of a plurality of openings, the plurality of openings disposed in a predetermined location to the at least one of the users mouth and the users nostril.

7. A device according to claim 1 wherein, the first pipe is at least one of attached to an inner surface of the shell, disposed within a filler material provided on the inner surface of the shell, and between a liner and the shell wherein the liner is disposed so as to between the shell and the users head.

8. A device according to claim 1 wherein,
at least one of the connector comprises a gimbal mount and the second pipe comprises a gimbal section, the at least one of allowing movement of the second pipe relative to the shell by predetermined degrees of rotation in two orthogonal axes.

9. A device comprising:
a vent piece for demountably attaching to a vent, the vent piece comprising at least one opening of a plurality of openings to couple a gas flow within the vent piece to outside the vent piece, wherein
the vent forms part of a shell comprising at least an outer layer covering a predetermined portion of a users head, a first pipe, a connector attached to a predetermined location of the shell and for coupling a first end of the first pipe to a second pipe, and the vent which is attached to a predetermined location of the shell and comprising a first inlet and a first outlet, the first inlet coupled to second end of the first pipe and the first outlet disposed in a predetermined location relative to at least one of a users mouth and a users nostril when the user wears the shell and none of the vent piece, the plurality of openings, and shell enclose the at least one of the users mouth and the users nostril.

10. A device according to claim 9 wherein,
the plurality of openings are disposed according to whether the vent is to be positioned relative to the users nostril or users mouth.

11. A device comprising:
a head gear comprising covering a predetermined portion of a users head and comprising at least an outer layer, an inlet port for accepting gas and at least an outlet port of a plurality of outlet ports for venting the gas;
a connector attached to the inlet port for coupling the device to the source of gas; wherein attaching at least one vent of a plurality of vents to the at least an outlet port provides a venting of the gas under pressure at a predetermined location relative to at least one of the users mouth and the users nostril and where none of the head gear and vent enclose the at least one of the users mouth and the users nostril.

12. A device according to claim 11 wherein,
the head gear further comprises an inner layer which in conjunction with the outer layer forms a bladder for the gas.

13. A device according to claim 12 wherein,
the head gear further comprises at least one of an inner layer which in conjunction with the outer layer forms a bladder for the gas and an inner layer and a filling that provides at least one pathway for the gas from the inlet port to the vent.
